(19) United States
(12) Patent Application Publication
(10) Pub. No.: US 2026/0355511 A1
(43) Pub. Date: Nov. 12, 2026

(54) AUTOMATIC ELECTRODE PAIR SELECTION OF CIRCULAR PHRENIC NERVE STIMULATION LEAD

(71) Applicant: Covidien LP, Mansfield, MA (US)

(72) Inventors: Lilian Kornet, Eijsden (NL); Abhijit Bhattacharya, Hyderabad (IN)

(21) Appl. No.: 19/138,368

(22) PCT Filed: Dec. 15, 2023
(86) PCT No.: PCT/IB2023/062775

§ 371 Date: Jun. 12, 2025

Related U.S. Application Data
(60) Provisional application No. 63/476,214, filed on Dec. 20, 2022.

(51) Int. Cl. A61N 1/36; A61N 1/05

(52) U.S. Cl. CPC A61N 1/36017; A61N 1/0551; A61N 1/36031

(57) ABSTRACT

Phrenic nerve stimulation systems and methods are disclosed. In an example, a phrenic nerve stimulation system includes a stimulation lead including multiple electrodes through which stimulation pulses may be delivered; and a stimulation source coupled to the stimulation lead, the stimulation source comprising at least one processor and memory storing instructions that, when executed by the at least one processor cause the system to perform operations. The operations include delivering a stimulation pulse via an initial subset of electrodes; detecting ineffective stimulation of a phrenic nerve from the delivered stimulation via the initial subset of electrodes; based on detecting the ineffective stimulation, delivering a stimulation pulse via a new subset of electrodes from the multiple electrodes of the stimulation lead; detecting effective stimulation of the phrenic nerve from the stimulation pulse via the new subset of electrodes; and based on detecting effective stimulation of the phrenic nerve, delivering subsequent simulation pulses via the new subset of electrodes.

This Application claims priority from U.S. Provisional Patent Application 63/476,214, filed 20 Dec. 2022, the entire content of which is incorporated herein by reference.

### BACKGROUND

Medical ventilator systems are commonly used to provide respiratory support to patients who require prolonged breathing assistance. Ventilators typically use positive pressure ventilation to deliver a controlled volume of breathing gases to the lungs of a patient. While lifesaving and valuable, in some patients the prolonged use of positive pressure ventilation may lead to additional health complications, such as atrophy of the diaphragm, ventilator-induced lung injury, contraction of a respiratory-related illness, and delay in ventilator weaning. To help mitigate these health complications and preserve the normal functioning of the diaphragm, a clinician may implement a procedure in which the diaphragm is indirectly stimulated using an intravenous stimulation lead. To have a therapeutic effect on the diaphragm and ultimately aid the recovery of a patient, this type of stimulation must be appropriately applied to the electrodes of the stimulation lead. It is with respect to this general technical environment that aspects of the present technology disclosed herein are directed.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In an aspect, the technology relates to a phrenic nerve stimulation system. The system includes a stimulation lead including multiple electrodes through which stimulation pulses may be delivered; and a stimulation source coupled to the stimulation lead, the stimulation source comprising at least one processor and memory storing instructions that, when executed by the at least one processor cause the system to perform operations. The operations include delivering a stimulation pulse via an initial subset of electrodes from the multiple electrodes; detecting ineffective stimulation of a phrenic nerve from the delivered stimulation via the initial subset of electrodes; based on detecting the ineffective stimulation, delivering a stimulation pulse via a new subset of electrodes from the multiple electrodes of the stimulation lead; detecting effective stimulation of the phrenic nerve from the stimulation pulse via the new subset of electrodes; and based on detecting effective stimulation of the phrenic nerve, delivering subsequent simulation pulses via the new subset of electrodes.

In an example, the stimulation source is a ventilator. In another example, the stimulation pulse delivered via the initial subset of electrodes is delivered according to stimulus parameters; and the stimulation pulse delivered via the new subset of electrodes is delivered according to the stimulus parameters. In yet another example, detecting the ineffective stimulation includes comparing one or more respiratory metrics, for diaphragm movement caused by the stimulation pulse via an initial subset of electrodes, to one or more thresholds. In still another example, the initial subset of electrodes and new subset of electrodes are positioned on a deformable segment of the stimulation lead.

In another aspect, the technology relates to a computer-implemented method for selecting electrodes of a stimulation lead. The method includes selecting a first pair of electrodes of the stimulation lead; delivering a first stimulation pulse via the first pair of electrodes; detecting ineffective stimulation of a phrenic nerve by the first stimulation pulse; based on detecting ineffective stimulation the phrenic nerve by the first stimulation pulse, selecting a second pair of electrodes of the stimulation lead; delivering a second stimulation pulse via the second pair of electrodes; detecting ineffective stimulation of the phrenic nerve by the second stimulation pulse; based on detecting ineffective stimulation the phrenic nerve by the second stimulation pulse, selecting a third pair of electrodes of the stimulation lead; and delivering a third stimulation pulse via the third pair of electrodes.

In an example, the second pair of electrodes are positioned on a first side of the first pair of electrodes, and the third pair of electrodes are positioned on an opposite side of the first pair of electrodes. In another example, the first stimulation pulse is delivered according to first stimulus parameters, and the second stimulation pulse is delivered according to second stimulus parameters, the second stimulus parameters having a greater intensity than the first stimulus parameters. In yet another example, the third stimulation pulse is delivered according to first stimulus parameters having a first intensity, and the method further includes detecting ineffective stimulation of the phrenic nerve by the third stimulation pulse; and based on detecting ineffective stimulation the phrenic nerve by the second stimulation pulse, delivering a fourth stimulation pulse via the third pair of electrodes, the fourth stimulation pulse being delivered according to second stimulus parameters having a greater intensity than the first stimulus parameters. In still another example, the method further includes detecting ineffective stimulation of the phrenic nerve by the fourth stimulation pulse; and based on detecting ineffective stimulation the phrenic nerve by the fourth stimulation pulse, selecting a fourth pair of electrodes. In still yet another example, the method further includes detecting effective stimulation of the phrenic nerve by the fourth stimulation pulse; and based on detecting effective stimulation the phrenic nerve by the fourth stimulation pulse, delivering subsequent stimulation pulses via the third pair of electrodes according to the second stimulus parameters.

In another aspect, the technology relates to a computer-implemented method for selecting electrodes of a stimulation lead. The method includes delivering a stimulation pulse via an initial subset of electrodes of the stimulation lead; detecting ineffective stimulation of a phrenic nerve from the delivered stimulation via the initial subset of electrodes; based on detecting the ineffective stimulation, delivering a stimulation pulse via a new subset of electrodes of the stimulation lead; detecting effective stimulation of the phrenic nerve from the stimulation pulse via the new subset of electrodes; and based on detecting effective stimulation of the phrenic nerve, delivering subsequent simulation pulses via the new subset of electrodes.

In an example, detecting the ineffective stimulation includes comparing one or more respiratory metrics, for diaphragm movement caused by the stimulation pulse via an initial subset of electrodes, to one or more thresholds. In another example, the initial subset of electrodes and new subset of electrodes are positioned on a deformable segment of the stimulation lead. In yet another example, the initial subset of electrodes are positioned on a first deformable segment of the stimulation lead and new subset of electrodes are positioned on a second deformable segment of the stimulation lead.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts the anatomy of a human patient, including phrenic nerves and a diaphragm.

FIGS. 2A-F depicts examples of a multielectrode endovascular stimulation lead in multiple configurations.

FIG. 2G depicts a cross-sectional view of a deformable segment of a stimulation lead with electrodes inside a blood vessel, in the vicinity of a phrenic nerve.

FIG. 3A-B depicts the anatomy of a human patient with placement of one or more stimulation leads.

FIG. 4A shows an example method for selecting electrodes for phrenic nerve stimulation.

FIG. 4B depicts another example method for selecting electrodes for phrenic nerve stimulation.

FIG. 4C depicts another example method for selecting electrodes for phrenic nerve stimulation.

FIG. 5 is a diagram illustrating an example of a ventilator connected to a human patient and a stimulation lead.

FIG. 6 is a block diagram illustrating an example of a ventilatory system.

### DETAILED DESCRIPTION

Medical ventilators are used to supply breathing gases to patients requiring respiratory support. Approximately one third of patients in the intensive care unit (ICU) depend on mechanical ventilation. With millions of people each year admitted to the ICU, many people per year rely on mechanical ventilation. The amount of time a patient is supported by mechanical ventilation may vary, based on the health condition of the patient. For example, approximately a third of patients with chronic obstructive pulmonary disease (COPD) or acute respiratory distress syndrome (ARDS) may seek assistance from a mechanical ventilator for longer than four days, with some patients requiring mechanical ventilation longer than seven days. Additionally, patients with sleep apnea may require mechanical ventilation while sleeping, and such support may occur over an extended period. Patients with other types of health conditions, such as patients with spinal cord injury or patients undergoing deep anesthesia, may also require ongoing respiratory support via mechanical ventilation.

Long term external ventilation is typically provided to patients using positive-pressure ventilation, where the ventilator is used to deliver a controlled volume of breathing gases to the lungs. While clinically valuable and often lifesaving, positive-pressure ventilation is not a natural physiologic breathing process, and in some patients the prolonged use of mechanical ventilation may lead to additional health complications. For example, in some cases, patients receiving higher numbers of positive-pressure cycles and higher positive pressures may be at greater risk of experiencing ventilator-induced lung injury (VILI), ARDS, ventilator-associated pneumonia (VAP), infection, and/or other respiratory illnesses. Further, prolonged use of positive-pressure ventilation may extend or worsen an existing illness, result in atrophy of the diaphragm, and/or delay ventilator weaning. These effects may increase the amount of time a patient is subjected to mechanical ventilation, leading to longer hospital stays and increased medical costs. Increased time on a ventilator may also be associated with increased risk of rehospitalization.

In contrast to positive-pressure ventilation, in one form of negative-pressure ventilation, the diaphragm is caused to contract, causing a negative pressure that draws air into the lungs (i.e., inhalation). When the diaphragm is caused to relax, the natural recoil of the lungs and other tissues causes air to be forced out of the lungs (i.e., exhalation). Negative-pressure ventilation is the process of normal or natural physiologic breathing. To mitigate the above complications associated with long-term positive-pressure ventilation, during ventilation or in lieu of ventilation, a patient's diaphragm may be exercised to induce negative-pressure ventilation. To exercise a patient's diaphragm, one or both phrenic nerves may be electrically stimulated. This electric stimulation may be provided by a stimulation source coupled to electrodes that are positioned close enough to one or both phrenic nerves to sufficiently stimulate the phrenic nerve(s) to cause negative-pressure ventilation via contraction of the diaphragm.

In examples, the electrodes may be positioned on one or more stimulation leads that are insertable into the body. For instance, the electrodes may be arranged on one or more stimulation leads that are inserted into blood vessels (e.g., veins, arteries, etc.) and positioned near one or both phrenic nerves that pass in the vicinity of the vessels. Regions within one or more vessels in which electrodes may be positioned to stimulate the phrenic nerve(s) may be referred to as stimulation sites.

Once electrodes are positioned at or near one or more stimulation sites, stimulation may be configured and applied to achieve a desired level of negative-pressure ventilation. For example, for each stimulation lead, an initial electrode pair or pairs may be selected from a plurality of electrodes spaced along various segments of each stimulation lead. In addition, configuring the stimulation may include selection of initial stimulus parameters such as stimulus pulse amplitude, pulse width, and/or other parameters may be selected. Respiratory metrics such as tidal volume, inspiratory flow, inspiratory pressure, and/or other metrics may be observed from the ventilator to gauge the effectiveness of the applied stimulation, and to guide initial adjustments to the stimulus configuration as needed. Following the initial configuration, if the position of a stimulation lead changes or drifts over time, the electrodes may be dislocated from the stimulation site(s), which may lead to reduced effectiveness or loss of stimulation therapy.

In some examples, to mitigate unintended movement of the stimulation lead, a method called active fixation may be used. With active fixation, an active/movable element of the stimulation lead is caused to become embedded or anchored in the wall of the vessel or other tissue, which may help maintain the stimulation lead in position. The disadvantage of active fixation is that when therapy is complete, retraction of the stimulation lead from the vessel may result in injury or damage to the vessel, and/or may result in other health risks.

Another method for securing a stimulation lead in a vessel is called passive fixation. With passive fixation the position of the lead is maintained in the vessel by passive features or characteristics of the lead, rather than by embedding an active/movable element within the wall of the vessel or other tissue. In one example, a stimulation lead may be held in place by friction between the interior wall of the vessel and portions of the surface of the stimulation lead. In another example, passive fixation may be achieved by designing the stimulation lead with a particular shape that attempts to prevent significant movement. In still other examples, the stimulation lead may employ passive elements, such as tines or other structures, that grip the interior wall of the vessel. In some instances, a stimulation lead may utilize a combination of several of these approaches to prevent unintended dislocation of the stimulation lead electrodes from the phrenic nerve stimulation site(s). One benefit of passive fixation is that the stimulation lead may be significantly easier to extract from the vessel and/or may reduce the risks associated with active fixation. However, stimulation leads that use passive fixation may be subject to significantly more unintended movement within the vessel than a stimulation lead that uses active fixation. With passive fixation, more types of motion of the patient may result in dislocation of the electrodes from the stimulation site(s).

Aspects of the present disclosure describe technology for automatic reconfiguration and selection of phrenic nerve stimulation electrodes that may mitigate the effects of unintended movement of passively fixated stimulation leads within a vessel. Some stimulation leads may have many electrodes from which a particular pair of electrodes may be selected. As discussed briefly above, when the stimulation lead is initially positioned, a first pair of electrodes are selected to provide the stimulation of the phrenic nerve. As the first selected pair of electrodes of a stimulation lead migrate away from a stimulation site due to motion of the patient, the effectiveness of the stimulation may be reduced or completely lost. This change in stimulation effectiveness is observable as a change to one or more of the respiratory metrics. For example, the tidal volume associated with the stimulation-induced negative-pressure ventilation may be reduced due to a first (initial) pair of electrodes shifting away from a stimulation site.

With the technology disclosed herein, in response to the reduction in tidal volume, the stimulation is automatically reconfigured such that the stimulus is applied to a second pair of electrodes that may have shifted towards the stimulation site or may otherwise be near enough to the stimulation site to provide effective stimulation. One or both of the electrodes of the second pair may be adjacent to one or both electrodes of the first pair. If the second pair of electrodes provides insufficient increase in tidal volume (or other respiratory metric), a third pair of electrodes may be automatically selected, where one or both of the electrodes of the third pair may be adjacent to one or both electrodes of the second pair. This selection pattern may continue until sufficient tidal volume is restored. In examples, electrodes may be automatically selected from either side of the first electrode pair, or selection may alternate from one side to the other, until the most effective electrode pair is determined. In some examples where multiple stimulation leads are implemented, shifts in the position of multiple stimulation leads may cause automatic reconfiguration of the stimulation (electrode selection and stimulus parameters) in both leads independently. In examples where both left and right phrenic nerves are stimulated, electrode selection may occur independently and simultaneously. Further details are now provided by way of discussion of the drawings.

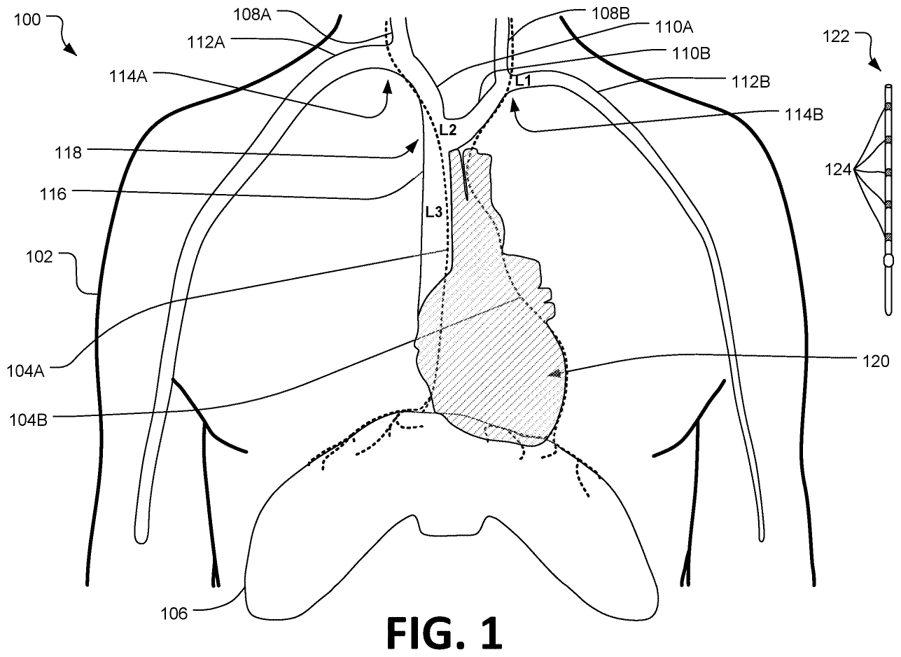

FIG. 1 depicts the anatomy of a human patient 100. The body 102 of the patient 100 includes a right phrenic nerve 104A, a left phrenic nerve 104B, a diaphragm 106, a right internal jugular vein 108A, a left internal jugular vein 108B, a right brachiocephalic vein 110A, a left brachiocephalic vein 110B, a right subclavian vein 112A, a left subclavian vein 112B, a right jugular-brachiocephalic junction 114A, a left jugular-brachiocephalic junction 114B, a superior vena cava (SVC) 116, an SVC junction 118, and a heart 120.

The right phrenic nerve 104A and the left phrenic nerve 104B originate from the spinal cord in the neck region (C3-C5 cervical vertebral region). The right phrenic nerve 104A extends through the body 102 from the right side of the neck region between the right lung and the heart 120 to the right side of the diaphragm 106. The right phrenic nerve 104A passes near or in the vicinity of portions of the right jugular-brachiocephalic junction 114A, the right brachiocephalic vein 110A, the SVC junction 118, and/or the SVC 116. The left phrenic nerve 104B extends through the body 102 from the left side of the neck region between the left lung and the heart 120 to the left side of the diaphragm 106. The left phrenic nerve 104B passes near or in the vicinity of portions of the left internal jugular vein 108B, the left jugular-brachiocephalic junction 114B, and/or the left brachiocephalic vein 110B. The right phrenic nerve 104A and the left phrenic nerve 104B may each, independently, cause muscle movement of the diaphragm 106 using an electrical signal conducted down the nerve. The phrenic nerve is “stimulated” when the phrenic nerve sends an electrical signal to the diaphragm 106. The phrenic nerve is “effectively stimulated” or “captured” when one or more muscles of the diaphragm 106 move (such as stiffening or contracting) due to the electrical signal conducted by the phrenic nerve.

In some examples, stimulation of the right phrenic nerve 104A causes one or more muscles on a right portion of the diaphragm 106 to move, thereby causing expansion of one or both lungs of the patient 100. In another example, stimulation of the left phrenic nerve 104B causes one or more muscles on a left portion of the diaphragm 106 to move, thereby causing expansion of one or both lungs of the patient 100. Stimulation of only one of the phrenic nerves (i.e., right phrenic nerve 104A or left phrenic nerve 104B) may cause muscle movement on both sides of the diaphragm 106, due to the collective nature of the diaphragm muscles (e.g., stimulating muscles on one side will also move some of the muscles on the other side). For some patients, stimulation of only one of the phrenic nerves is sufficient, while other patients may require or desire stimulation of both phrenic nerves. In some instances, one of the phrenic nerves may receive reduced or partial stimulation, whereby a portion of the diaphragm 106 is caused to stiffen, rather than fully contract, while the other phrenic nerve may receive full stimulation to fully contract the diaphragm 106. This approach may allow for more complete contractions of the diaphragm 106 with less stimulus energy. Further details are provided in in U.S. patent application Ser. No. 16/888,960, titled “Achieving Smooth Breathing by Modified Bilateral Phrenic Nerve Pacing,” and filed on Jun. 1, 2020, which is incorporated herein by reference in its entirety. The above application is referred to herein as the '960 application.

Stimulation of the phrenic nerve may originate naturally from the brain or may be provided artificially (e.g., by a nearby electrical current, and resulting voltages, from electrodes associated with a stimulation source). For example, the right phrenic nerve 104A and/or the left phrenic nerve 104B may be artificially stimulated by voltages resulting from an electrical current between two or more electrodes on a stimulation lead (such as an intravenous stimulation lead). For the induced voltages to effectively stimulate the phrenic nerve, the electrodes carrying the electrical current should be placed near or adjacent to the right phrenic nerve 104A and/or the left phrenic nerve 104B. In an example, a phrenic nerve may be stimulated by voltages induced by a current between two or more electrodes positioned inside the body of a patient, at a location where a phrenic nerve is near the resulting induced voltages. As described above, such a location may be referred to as a stimulation site.

FIG. 1 shows the approximate locations/regions of example stimulation sites L1, L2, and L3 accessible inside the body of the patient 102, within the described vasculature. Electrodes may be placed at or near stimulation site L1 to stimulate the left phrenic nerve, and electrodes may be placed at stimulation sites L2 and/or L3 to stimulate the right phrenic nerve. These locations may vary considerably from patient to patient, and the locations may vary within an individual patient if the body posture or position of that patient changes. Stimulation applied to pairs of electrodes located at one or more of these stimulation sites may induce voltages near enough to one or both phrenic nerves to stimulate one or both nerves, which may cause the diaphragm 106 to move or stiffen (e.g., contract). In some examples, other stimulation sites may be accessible within the vasculature.

In one example, an intravenous stimulation lead, such as stimulation lead 122, may be passed through the skin, and into the vasculature of the patient 102. The stimulation lead 122 may be inserted such that electrodes 124 are positioned at, or in the vicinity of, a stimulation site L1, L2, or L3, or at another potential stimulation site. A pair of electrodes may be selected from the set of electrodes 124 to stimulate a phrenic nerve. The electrode pair may be selected based on the response of the diaphragm 106 to applied stimulation. For instance, stimulation may be applied to different combinations of electrode pairings until a best electrode pair is determined, which may be based on evaluation of a respiratory metric such as tidal volume, for example.

The depictions of stimulation lead 122 and electrodes 124 in FIG. 1 are provided to aid visualization of intravenous phrenic nerve stimulation and are not intended to represent the physical dimensions or limitations of actual lead/electrode configurations. Additional details of example stimulation lead and electrode configurations are now described.

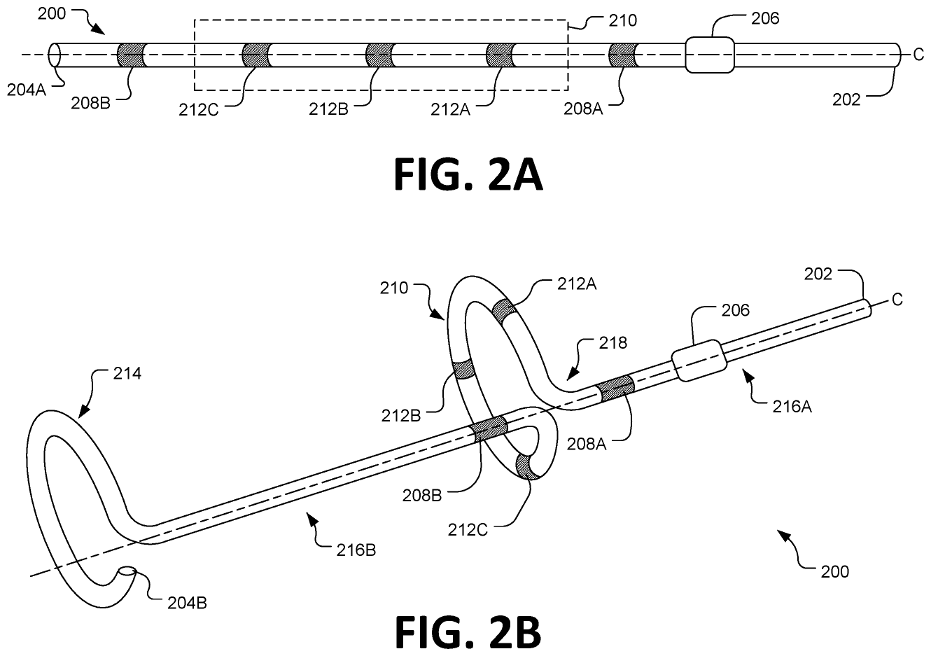

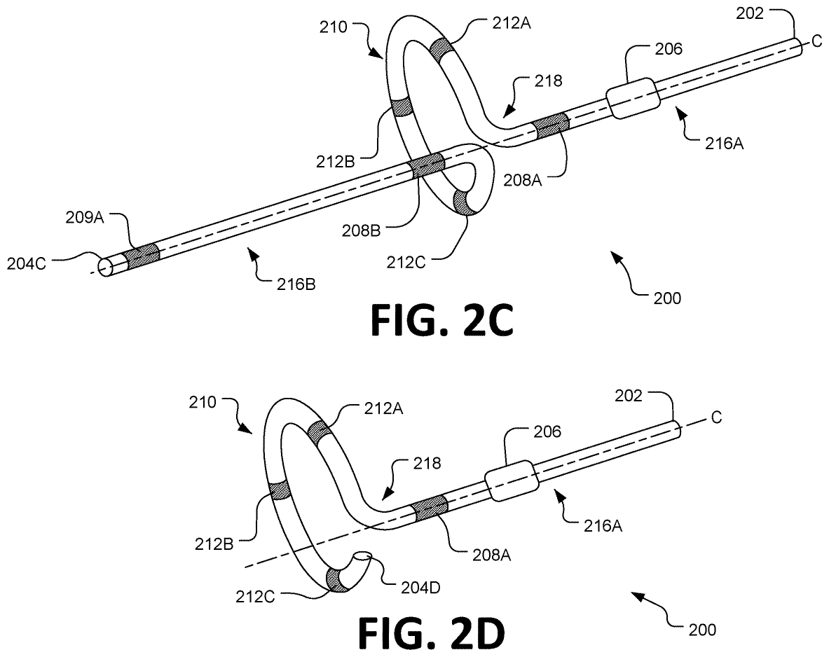

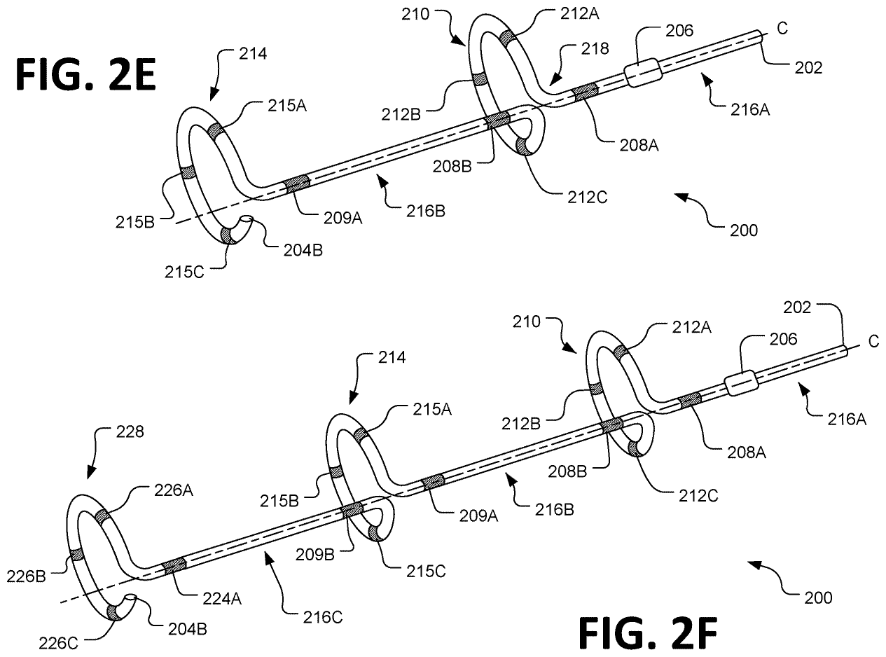

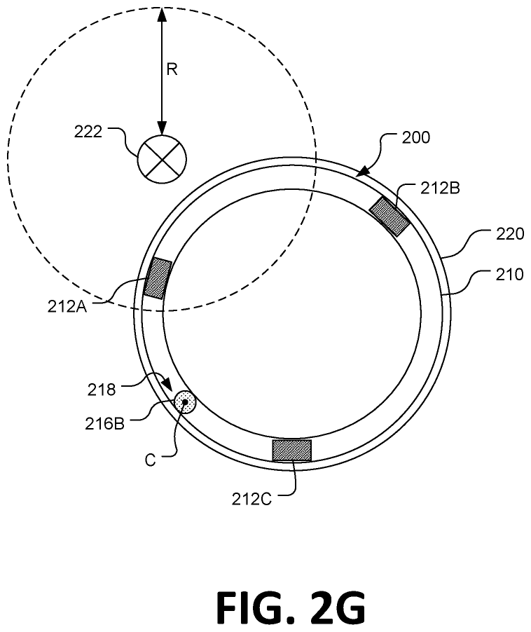

FIG. 2A-F show examples of an endovascular stimulation lead 200 in two configurations (e.g., an elongate configuration shown in FIG. 2A and shape configuration shown in FIGS. 2B-F), for phrenic nerve stimulation. As referred to herein, the stimulation lead 200 is in a shape configuration when one or more deformable segments 210, 214, 228 are in a non-elongate configuration. The examples shown depict a stimulation lead 200 in an elongate configuration that is substantially straight from the proximal end 202 to the distal end 204A. Additionally, as shown, the stimulation lead 200 in the shape configuration has one or more non-elongate segments (otherwise referred to as deformable segments) between the proximal end 202 and the distal end 204B-D. For example, deformable segments 210, 214, 228 are shown between the proximal end 202 and distal end 204B of FIGS. 2B, 2E, and 2F. Alternatively, FIGS. 2C-D show one deformable segment 210 between the proximal end 202 and the distal end 204C, 204D. A second elongate portion 216B may extend between the deformable segment 210 and the distal end 204C. One or more electrodes may be positioned on the second elongate portion 216B, while in other examples the second elongate portion 216B may not contain electrodes.

The stimulation lead 200 in the elongate configuration in FIG. 2A includes a proximal end 202, a distal end 204A, an optional fixation element 206, a first deformable segment 210, and electrodes 208A-B, 212A-C. The proximal end 202 may protrude from the body of the patient or may be inside the body. In an example, the first deformable segment 210 is positioned inside the body of the patient near or proximate a phrenic nerve (near a stimulation site, for instance). The distal end 204A extends past the phrenic nerve that is adjacent to the first deformable segment 210. A deformable segment is referred to herein as a portion of the stimulation lead that may transition from an elongate configuration to a non-elongate configuration. Different deformable segments may have different non-elongate configurations. As shown, the first deformable segment 210 includes electrodes 212A-C. Although three electrodes 212A-C are shown in the first deformable segment 210 of FIG. 2A, in examples, any number of electrodes may be included in the first deformable segment. Additionally, although one electrode 208A is shown along the first elongate segment 216A, in examples, any number of electrodes may be included in the first elongate segment 216A. Likewise, although one electrode 208B is shown along the second elongate segment 216B (of FIG. 2A-B), in examples, any number of electrodes may be included in the second elongate segment 216B.

In an example where one electrode is included in the first deformable segment 210, the electrode may be positioned about the first deformable segment 210 opposite the first elongate segment 216A and/or second elongate segment 216B of the stimulation lead 200 in the shape configuration. In another example, a single electrode of the first deformable segment 210 may be positioned opposite an electrode 208A, 208B on the first elongate segment 216A or second elongate segment 216B.

A second deformable segment 214 may not include electrodes, as shown in FIG. 2B. Additionally or alternatively, adjacent portions of the stimulation lead may not include electrodes. For example, a second elongate segment 216B of the stimulation lead coupled to the second deformable segment 214 may not include electrodes. Alternatively, the second deformable segment 214 may include electrodes 215A-C, as shown in FIG. 2E. In an example, the second deformable segment 214 may have the same or similar electrode configuration as first deformable segment 210. In another example, the second deformable segment 214 may include electrodes 215A-C in a different quantity and/or configuration than the first deformable segment 210 (e.g., 2-12 electrodes 215A-C). Additionally, one or more electrodes 208B, 209A may be positioned along the second elongate portion 216B between the first deformable segment 210 and the second deformable segment 214.

In some examples, the stimulation lead 200 may include three deformable segments as shown in FIG. 2F. A third elongate segment 216C of the stimulation lead, coupled to the third deformable segment 228, may include electrodes 209B and 224A, though in other examples, the third elongate segment 216C may include fewer or greater electrodes. In some examples the third deformable segment 228 may include electrodes 226A-C, while in other examples, fewer or greater numbers of electrodes may be included.

Although FIGS. 2B-F depict example stimulation leads with one, two, and three deformable segments, the stimulation lead may include any number of deformable segments with any shape at any point along the stimulation lead. Furthermore, both the deformable segments and elongate segments may have fewer or greater numbers of electrodes than depicted in FIGS. 2B-F. For example, deformable segments 210, 214, 228 may have 2-12 electrodes, which may be linearly spaced from one another, or may be spaced according to some other arrangement. Similarly, the elongate segments 216A-C of the stimulation lead may have 2-12 electrodes, which may be linearly spaced, or may be spaced according to some other arrangement.

Electrodes along the stimulation lead 200 may be made of an electrically conductive material. The deformable segments 210, 214, 228 of the stimulation lead 200 may be made of a shape-memory polymer capable of returning to an original shape after being deformed to a temporary shape. In an example, the non-elongate configuration of the deformable segments 210, 214, 228 may be the original shape of the deformable segments 210, 214, 228 and the elongate configuration may be the temporary shape. The shape-memory polymer may be induced into the temporary shape (e.g., the elongate shape) using a variety of inducers such as temperature, light, chemical agent, magnetic field, mechanical force, etc. For example, a stiff stylet may be inserted into an inner lumen of the stimulation lead 200 to straighten one or more deformable segments 210, 214, 228 into their elongate configuration. The stylet may be made of a stiff material such as stainless steel or tungsten. While the stylet is inserted into the stimulation lead 200, the stimulation lead 200 may be positioned inside the body of the patient. In an example, the stimulation lead 200 in the elongate configuration is placed inside the body proximate a phrenic nerve, while the stiff stylet is inserted into the stimulation lead 200. In further examples, the stimulation lead 200 may be placed within the vasculature of the patient, near or in proximity to one or both phrenic nerves. After removing the stylet from the stimulation lead 200 (otherwise referred to herein as pulling back the stylet, or retracting the stylet), the deformable segments 210, 214, 228 of the stimulation lead 200 return to the shape configuration. In the shape configuration, the deformable segments 210, 214, 228 may return to a substantially circular shape (as shown in FIGS. 2B-F) or may assume another shape. Additional details on stimulation lead shape configurations and deformable segments are provided in U.S. Patent Application Ser. No. 17/039,115, titled “Intravenous Phrenic Nerve Stimulation Lead,” and filed on Sept. 30, 2020, which is incorporated herein by reference in its entirety. The application will be referenced herein as the '115 application.

Once the stimulation lead 200 is placed in the body of the patient, such as in the vasculature of the patient, a passive fixation element 206 may help secure or maintain the stimulation lead 200 in position. For example, the passive fixation element 206 may increase friction between the stimulation lead 200 and the interior wall of a blood vessel or may maintain the position of the stimulation lead 200 by other passive methods. In some examples, the passive fixation element 206 may take the form of passive tines or other structures that grip the interior wall of the vessel. In other examples, the stimulation lead 200 may not include a passive fixation element 206.

The stimulation lead 200 in the elongate configuration may be positioned inside the body of the patient prior to transitioning the stimulation lead 200 to the shape configuration. For proper positioning, the stimulation lead may be moved inside the body while the stimulation lead provides an electrical current and resulting induced voltages, until the diaphragm muscles move due to stimulation of the phrenic nerve. In this regard, applying stimulation during placement of the stimulation lead may assist the process of initially positioning the electrodes at a stimulation site, sufficiently near or in proximity to one or both phrenic nerves. After positioning the stimulation lead 200 inside the body in the elongate configuration, one or more deformable segments of the stimulation lead 200 may be transitioned (e.g., by retracting a stylet), thereby resulting in the stimulation lead 200 transitioning into a shape configuration. One or more deformable segments may be configured to contact an interior wall of a blood vessel when in a shape configuration. For example, as shown in FIGS. 2B-F, the first deformable segment 210 is shaped into a substantially planar circle along the stimulation lead 200 in the shape configuration, which may provide one or more points of contact between the stimulation lead 200 and the interior wall of a vessel. Additional details of stimulation lead placement, transitioning, and examples of shape configurations are provided in the '115 application.

When in the shape configuration, electrodes placed within a deformable segment may be positioned to increase the likelihood that induced voltages will effectively stimulate the phrenic nerve. FIG. 2G depicts a cross-sectional view of a deformable segment 210 of the stimulation lead 200 with electrodes 212A-C inside a blood vessel 220, in proximity to a phrenic nerve 222. The blood vessel 220 may be a vein, artery, or other vessel. In a specific example, the blood vessel 220 is a vein (such as depicted in FIG. 1), and a portion of the vein is in proximity to a phrenic nerve 222. As shown, the deformable segment 210 of the stimulation lead 200 may be circular in shape to distribute electrodes 212A-C inside the blood vessel 220. In an example, the deformable segment 210 of the stimulation lead 200 may position one or more electrodes 212A-C adjacent to the interior wall of the blood vessel 220. For example, the deformable segment 210 has a non-elongate shape to position electrodes 212A-C radially inside the blood vessel 220, outward from the center line C of an elongate segment 216B at bend 218. In another example, the non-elongate shape of the deformable segment 210 is configured to position at least one of the electrodes 212A-C adjacent to the phrenic nerve 222 (e.g., closer to the phrenic nerve than another electrode on the stimulation lead 200).

The electrodes of a stimulation lead 200 may be independently communicatively coupled and/or electrically coupled to one or more controllers or stimulation sources or devices (not depicted in FIG. 2A-G). The stimulation source may be electrically coupled to a power source. A power source may be an external battery that may be replaceable and/or rechargeable, or the power source may be shared with an external device (e.g., a power source of a ventilator). In examples, the stimulation source may be an implantable stimulation device, an external stimulation device, a ventilator equipped with stimulation control features, or other stimulation source. For instance, the stimulation lead 200 may be electrically coupled to a ventilator that may provide a stimulus to one or both phrenic nerves via stimulation lead 200, while contemporaneously ventilating a patient.

As described herein, the act of applying or delivering stimulation is referenced in terms of delivering a controlled current pulse or set of pulses. The controlled current pulse or set of pulses may have identifiable stimulus parameters such as pulse amplitude (amount of current), pulse width (duration), frequency or frequency content, number of bursts, and/or other parameters that may be used to define pulsed stimulation. The stimulus parameters may also have an associated intensity level associated with the amount of energy delivered by the corresponding stimulation pulse. For instance, stimulus parameters with a greater pulse amplitude and/or pulse width have a greater intensity than stimulus parameters with a lower pulse amplitude and/or pulse width. A ventilator or other stimulation source may control the current of a stimulus pulse through appropriate circuitry designed for this purpose. The circuitry may direct current through selected electrodes by directly controlling the current itself or by controlling a voltage applied to the electrodes.

To cause an electrical current to flow between electrodes of stimulation lead 200, two or more electrodes may be assigned opposite polarities. When a voltage difference is applied across the electrodes by the stimulation source, current flows between the at least two electrodes. If the electrodes are sufficiently near or in proximity to a phrenic nerve, the voltages induced by the stimulus current may effectively stimulate the phrenic nerve. In one example, the phrenic nerve 222 may be effectively stimulated by the voltages resulting from a current running between electrodes 212A-C, when the current travels inside a stimulation radius R of the phrenic nerve 222. The current between two or more electrodes 212A-C may be increased or decreased as required or desired. An increase in current may increase the amount of stimulation of the phrenic nerve 222 and/or increase the stimulation radius of the phrenic nerve 222. A decrease in current may decrease the amount of stimulation of the phrenic nerve 222 and/or decrease the stimulation radius of the phrenic nerve 222. The amount of stimulation of the phrenic nerve 222 may be changed based on patient efforts and/or desired or determined tidal volume or another respiratory metric. A stimulation pulse along two or more electrodes 212A-C may be delivered over a pulse period to thereby cause stimulation of the phrenic nerve 222 during the pulse period. In an example, delivery of the stimulation pulse may be coordinated with delivery of an inhalation phase of ventilation provided by a mechanical ventilator (sometimes referred to as inhalation pacing).

In examples, electrodes of the stimulation lead 200 may be assigned to or selected as one of a wide variety of possible stimulation polarity configurations. For example, electrodes 208A-B, 212A-C that are positioned adjacent to each other along the length of the stimulation lead may be assigned as pairs having opposite polarities (positive and negative). Different electrode pairs may also be selected for each of the deformable sections. For instance, a first pair of electrodes may be selected for the first deformable segment 210, a second pair of electrodes may be selected for the second deformable segment 214, a third pair of electrodes may be selected for the third deformable segment 228.

An initialization may be performed to improve the effectiveness of the configuration of the applied stimulation. The initialization may test a variety of stimulus parameters and electrode configurations in variety of combinations. Adjustments to stimulus parameters and/or electrode selections may be based on one or more respiratory metrics, such as tidal volume, inhalation flow, inhalation pressure, or other respiratory metric observed during phrenic nerve stimulation. The initialization may be performed manually or automatically, using a program while monitoring the effort on the patient to determine effective settings.

In one example, an initialization may be conducted as follows. During or after placement of the stimulation lead in the body, stimulation may be separately applied to one or more different subsets of electrodes 212A-C (or other electrodes), and the effects of such stimulation are observed and stored. In some examples, the applied stimulation may use set or default stimulus parameters such as current pulse amplitude, pulse width, frequency, etc. In other examples, the stimulus parameters may be determined by varying the values of each parameter (e.g., sweeping the parameter values over an allowable range). After stimulation has been applied to the subsets of electrodes 212A-C, the effects of the stimulation on one or more respiratory metrics are compared. In an example, the comparison may include an amount of diaphragm muscle movement and/or relative tidal volumes cause by the phrenic nerve stimulation. A subset or pair of electrodes with the greatest effect (e.g., large amount of diaphragm muscle movement and/or large tidal volume), along with the corresponding stimulus parameters, are identified. The identified pair of electrodes 212A-C may be considered an initial pair of electrodes and may be used to stimulate the phrenic nerve. In an example, a subset of electrodes may be determined to effectively stimulate the phrenic nerve 222 when delivering a stimulation pulse (e.g., electrodes 212A and 212B or electrodes 212A and 212C).

As described herein, a phrenic nerve 222 is stimulated by voltages induced from an electrical current between two or more electrodes. Thus, the initial subset of stimulating electrodes may be based on the distance between one or more electrodes in the subset of electrodes and the phrenic nerve 222. For example, if an electrode is a distance less than or equal to the stimulation radius R (which may be based on the strength of the stimulation), then the phrenic nerve 222 may be effectively stimulated by a current traveling to that electrode 212A-C. In the example shown in FIG. 2G, one electrode 212A is within the stimulating radius R of the phrenic nerve 222. Thus, electrode 212A is positioned to effectively stimulate the phrenic nerve 222. For example, a stimulation pulse between electrode 212A and either 212B or 212C may stimulate the phrenic nerve 222. In the example shown, the most effective pair of electrodes to stimulate the phrenic nerve 222 is a stimulation pulse between electrodes 212A and 212B (the two electrodes positioned closest to the phrenic nerve) because the induced currents are generated closer to the phrenic nerve 222. In an example, electrodes 212A and 212B may have opposite polarities while the remaining electrode 212C does not participate stimulation., and the polarity assignments may be adjusted or selected as part of the initialization process The subset of electrodes selected as the most effective electrodes to stimulate the phrenic nerve(s) may include two or more electrodes. For example, the subset of electrodes may be two electrodes (i.e., an electrode pair), or may be a combination of three or more electrodes. As referred to herein, for ease of description, the subset of electrodes through which stimulation may be applied to one or more phrenic nerves is referred to as an electrode pair but may include two or more electrodes.

Data related to the configuration of stimulation lead 200, the stimulus parameters, initialization, corresponding respiratory metrics, and/or other data may be stored for later use by a ventilator or other stimulation source. For example, the number and location of possible selectable electrodes on a stimulation lead may be made available to, and/or stored on, the ventilator or other stimulation source. Similarly, stimulus parameters established during initialization and corresponding respiratory metrics (e.g., tidal volume resulting from stimulation-induced respiration) may be stored for later use, such as for comparing stimulation performance of alternatively selected electrodes.

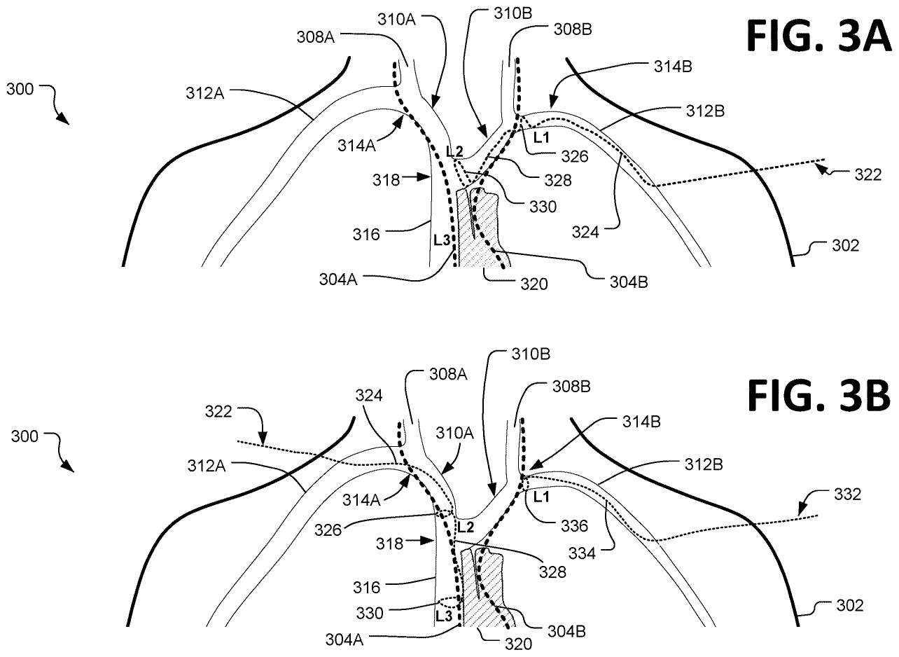

FIGS. 3A-B show the anatomy of a human patient 300 with intravenous placement of a first stimulation lead 322 and a second stimulation lead 332. The elements of the patient 300 may be similar to those described for the patient 100 in FIG. 1. As shown, the body 302 of the patient 300 includes a right phrenic nerve 304A, a left phrenic nerve 304B, a right internal jugular vein 308A, a left internal jugular vein 308B, a right brachiocephalic vein 310A, a left brachiocephalic vein 310B, a right subclavian vein 312A, a left subclavian vein 312B, a right jugular-brachiocephalic junction 314A, a left jugular-brachiocephalic junction 314B, a superior vena cava (SVC) 316, a SVC junction 318, and a heart 320.

The stimulation leads 322, 332 may have similar features to stimulation leads described herein (e.g., stimulation lead 200). FIG. 3A depicts an example of a single stimulation lead 322 that includes a first deformable segment 326 and second deformable segment 330, which may be the same or similar to deformable segments described herein (e.g., deformable segments 210, 214). The stimulation lead 322 may also include a first elongate segment 324 and a second elongate segment 328 which may each be the same or similar to elongate segments described herein (e.g., first elongate segment 216A and second elongate segment 216B).

As shown in FIG. 3A, the stimulation lead may be implanted in the vasculature of the body 302. A portion of the blood vessel in which at least a portion of the stimulation lead 322 is placed is proximal to a phrenic nerve (e.g., right phrenic nerve 304A and/or left phrenic nerve 304B). In the example shown, at least a portion of the stimulation lead 322 is placed in the left subclavian vein 312B in or near the left jugular-brachiocephalic junction 314B, near the left phrenic nerve 304B. A first deformable segment 326 of the stimulation lead 322 is located inside the left subclavian vein 312B, in or near the stimulation site LI for the left phrenic nerve 304B.

The stimulation lead 322 may also include a second deformable segment 330, as shown in FIG. 3A. The stimulation lead 322 may be similar to the shape configuration of the stimulation lead 200 shown in FIGS. 2B and 2E. The first deformable segment 326 and the second deformable segment 330 may be positioned any distance apart. In examples where the second deformable segment 330 includes one or more electrodes, the second deformable segment 330 may be placed at stimulation site L2 for stimulation of the right phrenic nerve 304A. In examples where the second deformable segment 330 does not include electrodes or otherwise does not participate in phrenic nerve stimulation, the second deformable segment 330 may be placed remotely from either phrenic nerve.

The first deformable segment 326 and the second deformable segment 330 may be coupled via the second elongate segment 328. One or more electrodes may be positioned on portions of the elongate segments 324, 328. These electrodes and electrodes of the deformable segments 326, 330 may be selected and configured for stimulation of one or both phrenic nerves.

Further, FIG. 3A depicts an example of a single stimulation lead inserted through the skin and into vasculature associated with the left side of the patient 300 (i.e., left subclavian vein 312B, left brachiocephalic vein 310B, etc.). In other examples, a single stimulation lead 322 may be inserted through the skin and into vasculature associated with the right side of the patient 300 (i.e., the right subclavian vein 312A, right jugular-brachiocephalic junction 314A, etc.). For example, a stimulation lead 322 may be inserted through the skin and into vasculature associated with the right side of the patient 300 and positioned such that electrodes of the stimulation lead 322 may stimulate the right phrenic nerve 304A at stimulation sites L2 and/or L3. In other examples, a single stimulation lead may be inserted through the skin and into vasculature associated with the left side of the patient 300, where the stimulation lead may position electrodes at stimulation sites L1, L2, and L3. For instance, a stimulation lead similar to the stimulation lead depicted in FIG. 2F (three deformable segments) may be inserted into the vasculature for achieving bilateral phrenic nerve stimulation. In still other examples, a single stimulation lead similar to any of those depicted in FIGS. 2B-F may be inserted into the body of the patient 300, for targeting one or more stimulation sites L1, L2, and/or L3.

FIG. 3B shows an example of dual stimulations leads 322, 332 inserted into the vasculature of the patient 300 for stimulation of both phrenic nerves 304A, 304B. Stimulation lead 332 includes a deformable segment 336 that may be similar to, or the same as, deformable segments described herein (e.g., deformable segment 210). The stimulation lead 332 may also include an elongate segment 334 that may be similar to, or the same as, elongate segments described herein (e.g., first elongate segment 216A). In the example shown, the stimulation lead 332 is inserted through the skin and into vasculature associated with the left side of the patient 300. In other examples, stimulation leads 322, 332 may be inserted on opposite sides of the body as shown in FIG. 3B. In some examples, stimulation leads 322, 332 may be any combination of stimulation lead configurations shown in FIGS. 2B-F or may be other configurations not depicted herein.

In both examples depicted in FIGS. 3A-B, and in other examples, the stimulation leads 322, 332 may be configured with electrodes positioned on elongate and/or deformable segments that enable stimulation of the left and/or right phrenic nerves 304A, 304B at stimulation sites L1, L2, and/or L3. In examples, the stimulation leads 322, 332 may be positioned in any blood vessel in the body 302 that runs proximate a phrenic nerve (e.g., the right internal jugular vein 308A, the left internal jugular vein 308B, the right brachiocephalic vein 310A, the left brachiocephalic vein 310B, the right subclavian vein 312A, the left subclavian vein 312B, the right jugular-brachiocephalic junction 314A, the left jugular-brachiocephalic junction 314B, SVC 316, SVC junction 318, carotid arteries, subclavian arteries, aorta, etc.).

As described above, data related to the configuration of stimulation lead 322 and/or 332, the stimulus parameters, initialization, corresponding respiratory metrics, and/or other data may be stored for later use by a ventilator or other stimulation source. For example, the number and location of possible selectable electrodes on a stimulation lead may be made available to, and/or stored on, the ventilator or other stimulation source. Similarly, stimulus parameters established during initialization and corresponding respiratory metrics (e.g., tidal volume resulting from stimulation-induced respiration) may be stored for later use, such as for comparing stimulation performance of alternatively selected electrodes.

Once inserted into the vasculature, stimulation leads 322, 332 may remain in the vasculature for a prolonged period, which may coincide with prolonged periods of ventilation. The inserted leads may be used to provide stimulation during discrete stimulation sessions, as part of a prescribed stimulation therapy regimen, designed to mitigate the health effects of prolonged positive-pressure ventilation (such as diaphragm atrophy). For example, a patient may undergo stimulation therapy on a daily basis, over a period of days or weeks, while receiving ventilator support. The stimulation leads may remain inserted in the vasculature during this period. As described above, active fixation stimulation leads may be held in place using an active fixation element associated with the stimulation lead. Passive fixation leads may be held in place by friction between elements of the stimulation lead and the interior walls of the vessel. In examples where the proximal end of the lead (such as proximal end 202) remains outside the body 302 of the patient 300, passive fixation leads may also be held in place by securing the proximal end of the lead to the body 302 of the patient 300. For example, the lead may be partially fixated to the exterior of the patient by means of medical tape or other method that secures the lead.

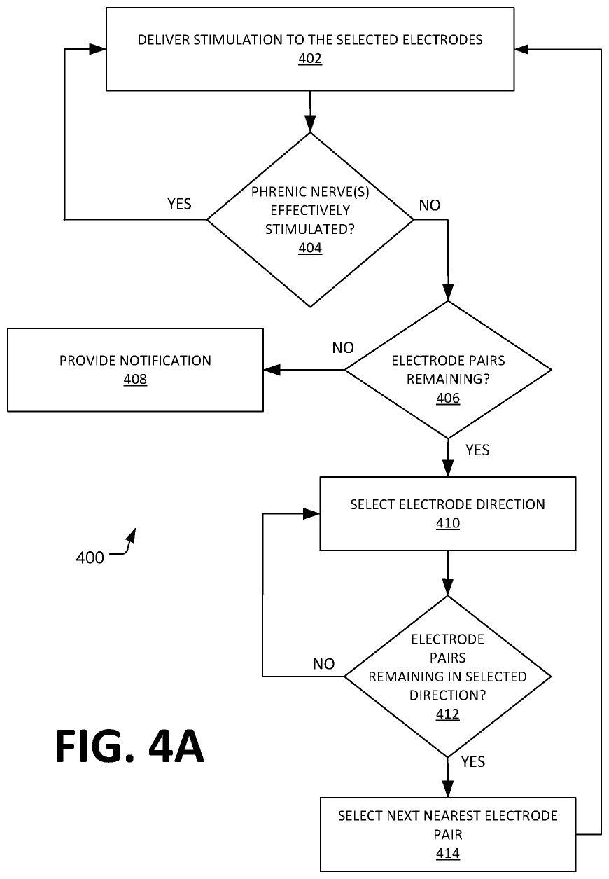

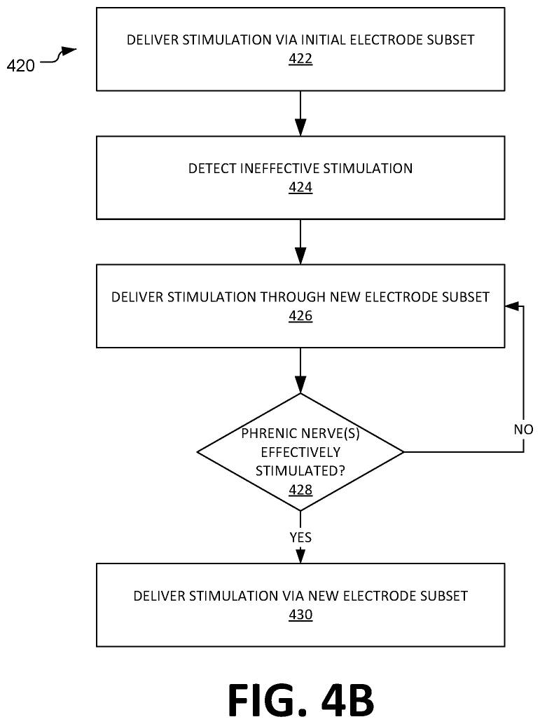

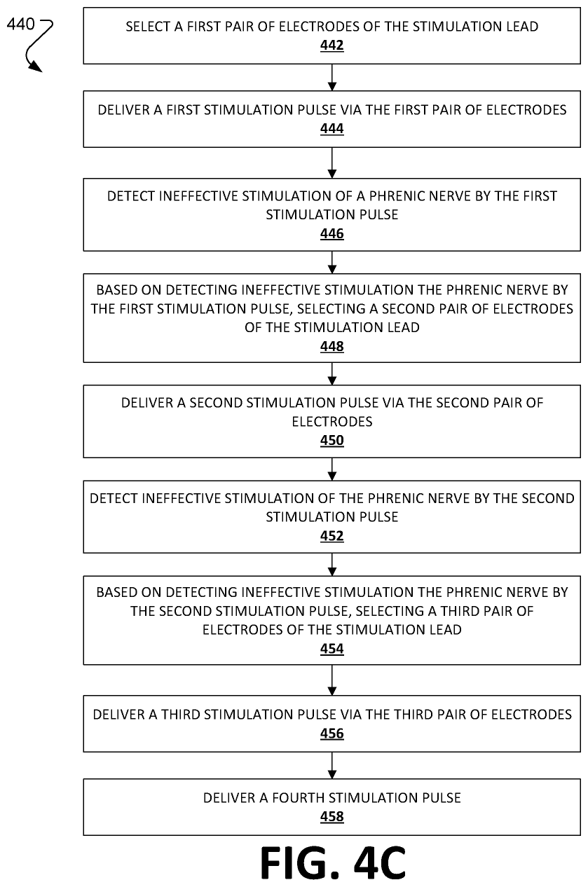

FIG. 4A shows an example method 400 for selecting electrodes for phrenic nerve stimulation. The example method 400 proceeds from an initial stimulation lead placement and initialization process. An example of such an initialization process is described above regarding FIG. 2. Briefly, one or more stimulation leads (such as stimulations leads 200, 322, 332) may be placed intravenously, with electrodes positioned at one or more stimulation sites (such as L1, L2, and/or L3) for stimulating one or both phrenic nerves. For each stimulation site where electrodes are positioned for stimulation, an electrode pair is selected along with stimulus parameters that optimize a respiratory metric (such as tidal volume, inhalation flow, inhalation volume, and/or other respiratory metrics) by effectively stimulating one or both phrenic nerves. Examples of stimulus parameters may include stimulation amplitude, pulse width, frequency, etc. Selection of the electrode pair may include assignment of electrode polarity. Once the initial electrode pair and stimulus parameters are selected that effectively stimulate one or both phrenic nerves, initialization is complete. The electrode pair, assigned polarities, stimulus parameters, respiratory metrics, and/or other data or information related to initialization may be stored for later use (such as in a memory element of the ventilator). In some examples, the process of initialization may be performed based on input or assistance from clinician. In other examples, the process of initialization may be performed automatically by an algorithm or computer program executing on a properly equipped ventilator or other stimulation device.

The example method 400 described herein may be performed during a stimulation session (such as at the beginning of a stimulation session) following initialization. In some examples, the stimulation leads may remain inserted in the vasculature of the patient, so that stimulation therapy may be provided to the patient over the course of a prolonged period of ventilation. This approach may mitigate the health effects described above (such as diaphragm atrophy), resulting from prolonged positive-pressure ventilation. The delivery of stimulation may take place during stimulation sessions, which may occur over a period of days or weeks, as part of a prescribed regimen of stimulation therapy. During this period when the stimulation leads remain inserted in the vasculature of the patient, the position of the stimulation leads (which may be passive fixation leads) may migrate, shift, or otherwise change. Consequently, the effectiveness of the applied stimulation may be reduced or completely lost. The example method 400 may help restore stimulation effectiveness due to shifts in stimulation lead position.

At operation 402, stimulation is delivered through the initial electrode pair. During the first performance of operation 402, the selected electrodes, assigned polarities, and stimulus parameters may be the same as those established by the initialization process and that were stored for use during subsequent stimulation sessions. For instance, operation 402 may include delivering a stimulation pulse via the initial electrode pair according to initial stimulus parameters in order to cause stimulation of a phrenic nerve.

The stimulation pulse may be triggered by a ventilator appropriately configured for phrenic nerve stimulation and electrically coupled to the electrodes of one or more stimulation leads. In other examples, the stimulation source may be an implanted device, an external stimulation device, or other type of device capable of phrenic nerve stimulation. In examples where the stimulation source is a device other than a ventilator, the stimulation source may be communicatively and/or operatively coupled to a ventilator for coordination of phrenic nerve stimulation, delivery of breathing gases to the patient, and/or feedback of respiratory metrics associated with the applied phrenic nerve stimulation. In examples, the stimulation may be automatically delivered in coordination with the respiratory functions of the ventilator. In one example, stimulation may be delivered to the phrenic nerve(s) in conjunction with a spontaneous breath attempted by the patient and detected by the ventilator. Upon detecting the attempted breath, the ventilator may apply stimulation to the phrenic nerve(s) via the electrodes of one or more intravenous stimulation leads. The stimulation may assist the patient in completing the breath. In examples where the patient is not capable of spontaneous breathing, the ventilator may apply stimulation in accordance with a prescribed breathing cadence or respiratory rate.

In some examples, delivering stimulation may include the bilateral stimulation techniques described in the '960 application, such as stiffening a first side of the diaphragm while fully stimulating the second (opposite) side of the diaphragm (which may reduce the stimulation energy required to cause contraction of the diaphragm). During subsequent breaths, the stimulation may be applied in the reverse order, where the second side is stiffened, and the first side is caused to contract. In other examples, the bilateral stimulation may include a delay between stimulation applied to one side of the diaphragm and stimulation applied to the other, such as to cause the lungs to fill asynchronously. In still other examples, one or both phrenic nerves may be effectively stimulated to cause contraction of one or both sides of the diaphragm, resulting in at least a partial inhalation.

At operation 404, a determination is made as to whether the phrenic nerve(s) are effectively stimulated from the stimulation pulse(s) delivered in operation 402. For instance, over time, a shift or change in the position of the stimulation lead, such as due to patient movement, may render the initially selected electrode pair ineffective The determination of effective stimulation may be at least partially based on one or more respiratory metrics observed by the ventilator and/or other monitoring devices. The respiratory metrics may include one or more of the tidal volume, inhalation flow, inhalation pressure, and/or other respiratory metrics. In some examples, the respiratory metrics observed by the ventilator during a stimulation session may be compared to respiratory metrics observed during the initialization process. For instance, the tidal volume observed during an ongoing stimulation session may be compared to the tidal volume established during initialization, which was determined to be an indication of effective stimulation of the phrenic nerve(s). A decrease in the tidal volume below a predetermined threshold may indicate an unacceptable reduction or loss of effective stimulation, relative to the stimulation applied during initialization. Similarly, other respiratory metrics observed during an ongoing stimulation session may be compared to metrics established and stored during initialization. One or more thresholds may be used to determine unacceptable changes or degradations in the respiratory metrics.

In another example, the inhalation flow may be observed over the course of a stimulation-induced inhalation or partial inhalation, or the inhalation flow may be observed over a series of breathing cycles (i.e., the inhalation flow may be observed over a period time). For instance, the gradient or slope of an inhalation flow curve may be evaluated for effective stimulation. Abrupt changes in the slope of the flow curve may indicate a brief, sharp inhalation (similar to a hiccup) rather than a longer, smooth inhalation. In a similar example, the inhalation rise time may be used to evaluate effective stimulation, where short duration of inhalation flow or pressure may indicate inadequate respiration by the patient.

The respiratory metrics observed by the ventilator may be compared to a threshold or set of thresholds to determine whether effective stimulation has been delivered. For example, the maximum tidal volume observed during a stimulation-induced inhalation, or over a plurality of breathing cycles observed over time, may be compared to a threshold. The threshold may be predetermined by a clinician, may be based on clinical data available to the ventilator, and/or may be based on some other relevant criteria. Additionally or alternatively, respiratory metrics such as inhalation flow, inhalation pressure, and/or other respiratory metrics may each be compared to an appropriate threshold to help determine whether one or more phrenic nerves have been effectively stimulated.

In another example, a pressure-volume (PV) curve may be developed over one or more stimulation-induced breathing cycles and used to evaluate effectiveness of the stimulation. For instance, a PV curve may indicate increasing inhalation pressure while inhalation volume stays constant (which may be referred to by some as “beaking”). A PV curve exhibiting this type of response may indicate ineffective stimulation. In examples, the PV curve may be evaluated for other indications of effective/ineffective stimulation, such as hysteresis, slope, peak, flatness, maximum pressure, maximum volume, or other measure. In some instances, the PV curve may be established over a plurality of breathing cycles, and/or the average PV curve may be evaluated for effectiveness of stimulation.

In another example, a flow-volume (FV) curve may be established over one or more stimulation-induced breathing cycles and used to evaluate effectiveness of the stimulation. For instance, the FV curve may be evaluated for hysteresis, slope, peak, flatness, maximum flow, maximum volume, or other measure. In some instances, the FV curve may be established over a plurality of breathing cycles, and/or the average FV curve may be evaluated for effectiveness of stimulation.

The ventilator or other stimulation source may receive additional measurement data associated with patient effort, movement of the diaphragm, or other indicators of stimulation effectiveness. For example, the ventilator may receive transthoracic impedance data, which may be correlated with aspects of respiration. For instance, the transthoracic impedance may increase/decrease in relation to chest expansion/contraction associated with a stimulation-induced breath. The slope of the transthoracic impedance over time may indicate abnormalities such as a hiccup or other rapid contraction of the diaphragm, which may be indicative of ineffective stimulation. The magnitude of the transthoracic impedance may be correlated with the volume of inhaled and exhaled breathing gases, which may indicate ineffective stimulation.

If one or both phrenic nerves have been found to have been effectively stimulated, flow may proceed “YES” to operation 402, for continuation of stimulation therapy (if appropriate). If it is determined that one or more phrenic nerves have not been effectively stimulated, flow proceeds “NO” to operation 406. At operation 406, a determination is made as to whether electrode combinations remain that have not been used to deliver phrenic nerve stimulation during the present stimulation session. At the initial performance of operation 406, the electrode pair being used to deliver stimulation is the same as the electrode pair selected during initialization. Accordingly, in the initial performance of method 400, operation 406 may be skipped or omitted.

To assist in performing method 400 and operation 406, a record of electrode pairs that have been used to deliver stimulation in the current stimulation session are stored, along with a record of all possible electrode pairs for the stimulation lead or leads being used with the patient. In some examples, the set of possible electrodes may consist of all possible pairings or combinations of electrodes of a stimulation lead. In other examples, the set of possible electrodes may be limited to a subset of all possible pairings or combinations of electrodes. As different electrode pairs are selected and used to deliver stimulation, the stored record is updated. If all possible electrode pairs or combinations have been used to deliver stimulation and none have been found to effectively stimulate the phrenic nerve(s), method 400 proceeds “NO” to operation 408.

At operation 408, a notification is generated. The notification may indicate that the stimulation lead(s) are not able to effectively stimulate the phrenic nerve(s). The inability to effectively stimulate the phrenic nerve(s) using electrodes on one or more stimulation leads may have a variety of causes. For instance, the inability to effectively stimulate the phrenic nerve(s) may result from damage to one or both phrenic nerves. In other examples, the inability to stimulate the phrenic nerve(s) may result from a change in the position of a stimulation lead, such that none of the electrodes on that lead are near enough to the phrenic nerve(s) to effectively stimulate the phrenic nerve(s). This type of situation may require the attention of a clinician, who may attempt to reposition one or more of the stimulation lead(s) to achieve effective stimulation.

The notification may be provided by visual and/or audio indicators associated with the ventilator or stimulation source. For example, the notification may be provided through a display coupled to, or integrated in, a ventilator or other stimulation source. Additionally or alternatively, the notification may be provided by a visual indicator such as a light, LED, or other discreet indicator. In examples, the notification may include an audible alert.

At operation 406, if pairs or combinations of electrodes remain that have not been used to deliver stimulation to the phrenic nerve(s) during the current stimulation session, flow proceeds “YES” to operation 410. At operation 410, a direction is selected in which a new electrode pair may be chosen to apply stimulation to the phrenic nerve(s). For example, electrodes may be selected from a set of one or more electrodes positioned distally from the currently selected electrode pair or from a set of one or more electrodes positioned proximally from the currently selected electrode pair. FIGS. 2A-B provide a visual reference for electrodes positioned distally/proximally to one another on stimulation lead 200. In one example, if a first electrode pair is composed of electrodes 212A and 212B, and a distal direction is chosen from which to select a second, subsequent pair of electrodes, electrode pair 212B and 212C may be selected, since this pair is distal to the first electrode pair. While electrodes 212A-C are located on a deformable segment, reference to distal/proximal may be understood by visualization of these electrodes with the stimulation lead 200 in the elongate configuration of FIG. 2A.

Selection of a direction in which to choose new electrodes for stimulation may be based on the configuration of the ventilator or other stimulation source. For example, on the first performance of operation 410, the configuration may indicate a default direction in which to select new electrodes. For instance, the configuration may indicate that one or more electrodes should be selected from the remaining set of electrodes positioned distally/proximally from the first selected electrodes (e.g., from the electrode pair or combination selected at initialization). In subsequent executions of operation 410, electrodes may continue to be selected from the remaining set of electrodes positioned still more distally/proximally from the currently selected electrodes, until no electrodes remain in that direction. Electrodes may then be selected from the set of electrodes remaining in the opposite direction from the first set of electrodes.

In other examples, selection of a direction in which to choose the next electrode pair or combination may be based on previous performance of operations of example method 400. For instance, previous performance(s) of operations of example method 400 may have resulted in selection of a second electrode pair positioned adjacent to, and more distally from, the first selected electrodes, and this second electrode pair may have effectively stimulated the phrenic nerve(s). However, the position of the stimulation lead containing the second electrode pair may have subsequently changed, and the second electrode pair may no longer effectively stimulate the phrenic nerve(s). The next electrode pair may again be selected from the remaining set of electrodes positioned distally from the second electrode pair, since previous performance of operation 410 was (temporarily) successful in restoring effective stimulation when the distal direction was chosen.

In still other examples, the selected direction in which to choose new electrodes may alternate between performances of operation 410. For instance, during a first performance of operation 410, a first direction may be selected (such as the proximal direction). During a subsequent performance of operation 410, the opposite direction may be selected (such as the distal direction). The selected direction may continue to alternate between the directions during additional performances of operation 410. In other examples, the direction may be selected from a plurality of possible selection patterns. The currently selected direction and previously selected direction may be stored (such as in a memory element of the ventilator) for use in subsequent performance(s) of operation 410.

At operation 412, a determination of the remaining electrode pairs in the selected direction is made. The determination may be made based on whether one or more electrodes are positioned further in the selected direction than the currently selected electrode pair. For example, if a distal direction is chosen from which to select a subsequent electrode pair, and no additional electrodes are positioned distally from the current electrode pair, flow proceeds “NO” to operation 410. The direction opposite to the current direction is then selected, which in this example would result in the proximal direction being selected. At operation 412, if one or more electrodes are positioned further in the selected direction than the currently selected electrode pair, flow proceeds “YES” to operation 414.

At operation 414, an electrode pair nearest the currently selected electrode pair (in the selected direction) may be selected to deliver stimulation. Returning to a previous example, referencing FIGS. 2A-B, if the currently selected (first) electrode pair is 212A-B, and the distal direction is chosen from which to select the next nearest electrodes, electrode pair 212B-C may be selected as the next (second) electrode pair. In a similar fashion, if the proximal direction is chosen (or if there are no remaining electrodes in the distal direction), electrode pair 212A-208A may be selected as the next electrode pair, since 212A-208A is the next nearest proximal electrode pair to the first electrode pair.

In some examples, the selected electrode pair may not be the next nearest electrode pair from the currently selected pair. For instance, clinical, heuristic, or other patient data may indicate an alternative approach to electrode selection, such as skipping to the second nearest electrode pair (i.e., every other nearest pair). In other examples, other selection patterns or approaches may be used to select the next electrode pair. In some examples, the next electrode pair may also be an electrode pair on another (e.g., second) stimulation lead that is inserted into the patient. Accordingly, electrode pairs across multiple stimulation leads may be tested.

Upon selection of a new pair of electrodes, flow proceeds back to operation 402, where stimulation is delivered with the newly selected pair of electrodes. Operation 404 is then where effectiveness of stimulation is again evaluated. The flow will proceed through the remaining operations as described. Performance of example method 400 may be repeated until effective stimulation is restored, or until notification is provided that the stimulation lead(s) cannot effectively stimulate the phrenic nerve(s).

In some examples, stimulus parameters may be varied during or between one or more operations of example method 400. For instance, if the phrenic nerve(s) are not (or are no longer) effectively stimulated as determined at operation 404, one or more stimulus parameters (such as stimulus amplitude, pulse width, etc.) may be automatically varied by the ventilator to attempt to restore effective stimulation. In examples, the stimulus parameters may be varied once all electrode pairs or combinations have been evaluated and found not to provide effective stimulation. In such examples, the ventilator may return to previously tried pairs of electrodes and vary the stimulus parameters to attempt to restore effective stimulation. In other examples, the stimulus parameters may be varied whenever a new electrode pair or combination is selected and found not to provide effective stimulation, before selecting the next pair or combination of electrodes.

FIG. 4B depicts another example method 420 for selecting electrodes for phrenic nerve stimulation. At operation 422, stimulation is delivered (e.g., a stimulation pulse) via an initial electrode subset of the electrodes of a stimulation lead. Such stimulation may be delivered as part of an ongoing stimulation therapy. At some point, potentially due to movement of the patient, the stimulation lead moves or rotates within the body (e.g., within the vessel), and at operation 424, ineffective stimulation of the phrenic nerve is detected. The ineffective stimulation may be determined by comparing one or more respiratory metrics to one or more corresponding thresholds. For instance, a decrease in tidal volume generated from stimulation to a level below a corresponding threshold may indicate an ineffective stimulation.

Based on detecting the ineffective stimulation at operation 424, a new electrode subset of the stimulation lead is selected and stimulation (e.g., a stimulation pulse) is delivered through the new electrode set at operation 426. In examples where multiple stimulation leads are positioned within the patient, the new electrode subset may be on the same or different stimulation lead as the initial or prior subset of electrodes.

At operation 428, a determination is made as to whether the stimulation delivered in operation 424 was effective. If the stimulation was effective, method 420 flows to operation 430 where subsequent stimulation is delivered via the new subset of electrodes used in operation 426. For instance, phrenic nerve stimulation continues with the new electrode subset. If, at operation 428, the stimulation is determined to be ineffective, the method 420 flows back to operation where yet another new electrode subset of electrodes of the stimulation lead is selected and stimulation is delivered via the new subset of electrodes. The method 420 continues in such a manner until all subsets of electrodes are tested or a subset of electrodes are identified that provide effective stimulation.

FIG. 4C depicts another example method 440 for selecting electrodes for phrenic nerve stimulation. At operation 442, a first pair of electrodes of a stimulation lead are selected. At operation 444, a first stimulation pulse is delivered via the first pair of electrodes. At operation 446, ineffective stimulation of a phrenic nerve by the first stimulation pulse is detected. Based on detecting ineffective stimulation of the phrenic nerve by the first stimulation pulse in operation 446, a second pair of electrodes of the stimulation lead are selected in operation 448.

At operation 450, a second stimulation pulse is delivered via the second pair of electrodes. At operation 452, ineffective stimulation of the phrenic nerve by the second stimulation pulse is detected. Based on detecting ineffective stimulation of the phrenic nerve by the second stimulation pulse, a third pair of electrodes of the stimulation lead are selected at operation 454. At operation 456, a third stimulation pulse is delivered via the third pair of electrodes.

At operation 458, a fourth stimulation pulse is delivered. The characteristics of the fourth stimulation pulse and/or the electrodes through which the stimulation pulse is delivered may be based on whether the third stimulation pulse effectively stimulated the phrenic nerve. If the third stimulation pulse effectively stimulated the phrenic nerve, the fourth stimulation pulse may be delivered via the third pair of electrodes based on the same stimulus parameters as the third stimulation pulse. If the third pulse did not effectively stimulate the phrenic nerve, the fourth stimulation pulse may be delivered via fourth pair of electrodes and/or based on different stimulus parameters as the third stimulation pulse. For instance, the stimulus parameters of the fourth stimulation pulse may have an intensity greater than the stimulus parameters used for delivering the third stimulation pulse.

The example methods 400, 420, 440 may be performed automatically by a ventilator or other stimulation source. However, in some examples, one or more operations of the example method 400, 420, 440 may be performed by a clinician. For instance, selection of a direction (proximal/distal) and electrode pair may be performed automatically, while effective stimulation may be determined/evaluated by a clinician, rather than automatically by the ventilator. Further, a clinician may perform adjustments to one or more aspects of the stimulation therapy. For example, a clinician may adjust stimulus parameters (such as stimulus amplitude, pulse width, frequency, etc.) based on evaluation of the response of a ventilated patient to the applied stimulation. In other examples, a clinician may select electrode pairs and/or stimulus parameters that produce effective stimulation, but are not optimal, based on evaluation of the patient response. For instance, in situations where multiple electrode pairs or combinations are found to effectively stimulate a phrenic nerve, a clinician may choose a pair that provides effective, but not most-effective, stimulation, based on response of the patient, health condition of the patient, and/or other considerations.

In some examples, the ventilator or other stimulation source may automatically initiate and perform example method 400, 420, 440 on a regular and/or scheduled basis, such as one or more times daily, based on a prescribed regimen of stimulation therapy. In other examples, a clinician may initiate performance of example method 400, 420, 440 at other times, such as between scheduled stimulation sessions or in the absence of a prescribed stimulation therapy schedule.

The ventilator or other stimulation source may store and use stimulation-related data associated with a patient. For instance, the ventilator may store data related to electrode pair selection, direction for next pair selection (proximal/distal), stimulus parameters, respiratory parameters, and/or other data related to delivery of stimulation to one or more phrenic nerves. In some examples, the data may be stored separately from the ventilator, such as on a computer system associated with a clinic. In this regard, the data may be accessible across multiple ventilators or other stimulation sources used to provide stimulation therapy to the patient.

The example method 400, 420, 440 may be performed with stimulation leads that are either active fixation leads or passive fixation leads. While active fixation leads are at a lower risk of movement within a blood vessel than passive leads, active fixation leads may still be prone to dislocation. For example, the active element of an active fixation lead may become detached from the wall of the vessel or other tissue and subject the lead to greater risk of movement within the vessel.

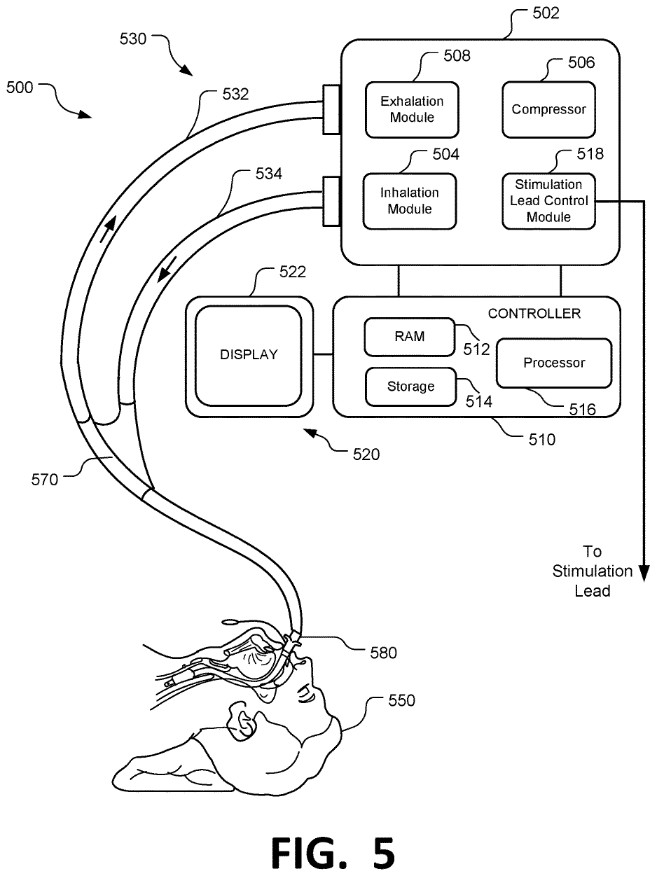

FIG. 5 is a diagram illustrating an example of phrenic nerve stimulation system in the form of a ventilator 500 connected to a human patient 550 and a stimulation lead. Ventilator 500 includes a pneumatic system 502 (also referred to as a pressure generating system 502) for circulating breathing gases to and from patient 550 via the ventilation tubing system 530, which couples the patient to the pneumatic system via an invasive (e.g., endotracheal tube, as shown) or a non-invasive (e.g., nasal mask) patient interface.

Ventilation tubing system 530 may be a two-limb (shown) or a one-limb circuit for carrying gases to and from the patient 550. In a two-limb example, a fitting, typically referred to as a “wye-fitting” 570, may be provided to couple a patient interface 580 to an inhalation limb 534 and an exhalation limb 532 of the ventilation tubing system 530.

Pneumatic system 502 may have a variety of configurations. In the present example, system 502 includes an exhalation module 508 coupled with the exhalation limb 532 and an inhalation module 504 coupled with the inhalation limb 534. Compressor 506 or other source(s) of pressurized gases (e.g., air, oxygen, and/or helium) is coupled with inhalation module 504 to provide a gas source for ventilatory support via inhalation limb 534. Stimulation lead control module 518 may provide voltage/current to a stimulation lead as described herein. The pneumatic system 502 may include a variety of other components, including mixing modules, valves, sensors, tubing, accumulators, filters, etc.

Controller 510 is operatively coupled with pneumatic system 502, signal measurement and acquisition systems, and an operator interface 520 that may enable an operator to interact with the ventilator 500 (e.g., change ventilator settings, select operational modes, view monitored parameters, etc.). Controller 510 may include memory 512, one or more processors 516, storage 514, and/or other components of the type found in command-and-control computing devices. In the depicted example, operator interface 520 includes a display 522 that may be touch-sensitive and/or voice-activated, enabling the display 522 to serve both as an input and output device.

The memory 512 includes non-transitory, computer-readable storage media that stores software that is executed by the processor 516 and which controls the operation of the ventilator 500. In an example, the memory 512 includes one or more solid-state storage devices such as flash memory chips. The processor 516 may be configured to control attributes of the electrodes on a stimulation lead. In an alternative example, the memory 512 may be mass storage connected to the processor 516 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 516. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer. For example, memory 512 may be used to store stimulation-related data, such as stimulation lead configuration (e.g., number of deformable segments, number of elongate segments, number and position of electrodes, etc.), stimulus parameters, respiratory metrics used for evaluation of stimulation, thresholds, and/or other data Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra-or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

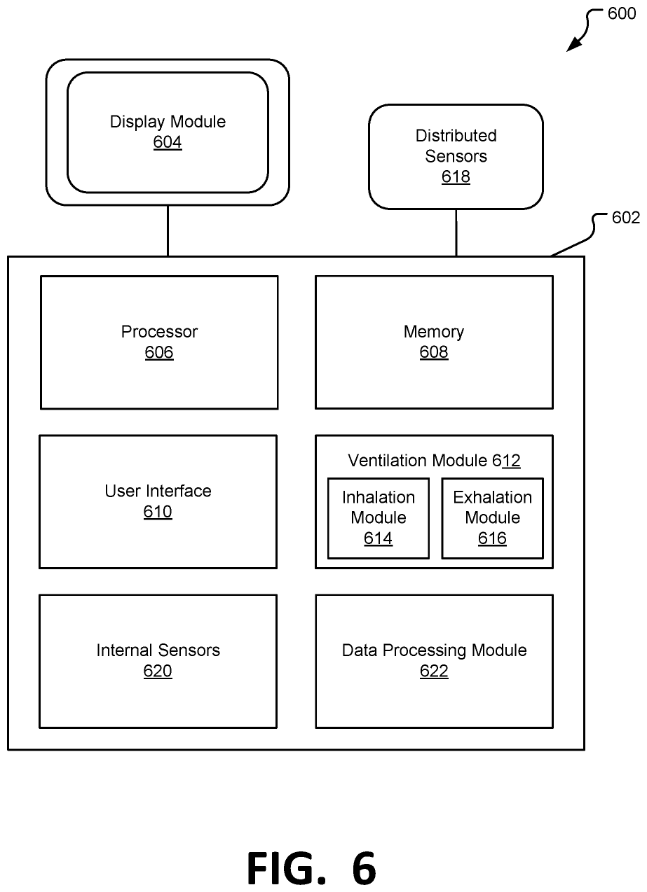

FIG. 6 is a block-diagram illustrating an example of a ventilatory system 600. Ventilatory system 600 includes ventilator 602 with its various modules and components. That is, ventilator 602 may further include, among other things, memory 608, one or more processors 606, user interface 610, and ventilation module 612 (which may further include an inhalation module 614 and an exhalation module 616). Memory 608 is defined as described above for memory 512. Similarly, the one or more processors 606 are defined as described above for one or more processors 516. Processors 606 may further be configured with a clock whereby elapsed time may be monitored by the system 600. The ventilator 602 may be connected to distributed sensors 618, such as sensors used to observe respiratory metrics such as flow, pressure, and/or other metrics. Internal sensors 620 may also be included in the ventilator 602.

The ventilation system 600 may also include a display module 604 communicatively coupled to ventilator 602. Display module 604 provides various input screens, for receiving input, and various display screens, for presenting useful information. Inputs may be received from a clinician. The display module 604 is configured to communicate with user interface 610 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 602 may be provided, for instance by a wheel, keyboard, mouse, or other suitable interactive device. Thus, user interface 610 may accept commands and input through display module 604. Display module 604 may also provide useful information in the form of various ventilatory data regarding the physical condition of a patient and/or a prescribed respiratory treatment. The useful information may be derived by the ventilator 602, based on data collected by a data processing module 622, and the useful information may be displayed in the form of graphs, wave representations (e.g., a waveform), pie graphs, numbers, or other suitable forms of graphic display. For example, the data processing module 622 may be operative to determine ventilation settings (otherwise referred to as ventilatory settings, or ventilator settings) associated with a nerve stimulation lead, etc., as detailed herein.

Ventilation module 612 may oversee ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with stimulating a phrenic nerve with a stimulation lead. In some cases, certain ventilation settings may be adjusted based on the exhalation flow, e.g., to optimize the prescribed treatment.

Ventilation module 612 may further include an inhalation module 614 configured to deliver gases to the patient and an exhalation module 616 configured to receive exhalation gases from the patient, according to ventilation settings that may be based on the exhalation flow. As described herein, inhalation module 614 may correspond to the inhalation module 504 and 614 or may be otherwise coupled to source(s) of pressurized gases (e.g., air, oxygen, and/or helium), and may deliver gases to the patient. As further described herein, exhalation module 616 may correspond to the exhalation module 508 and 616 or may be otherwise coupled to gases existing the breathing circuit.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A phrenic nerve stimulation system comprising:
a stimulation lead including multiple electrodes through which stimulation pulses may be delivered; and
a stimulation source coupled to the stimulation lead, the stimulation source comprising at least one processor and memory storing instructions that, when executed by the at least one processor cause the system to perform operations comprising:
delivering a stimulation pulse via an initial subset of electrodes from the multiple electrodes;
detecting ineffective stimulation of a phrenic nerve from the delivered stimulation via the initial subset of electrodes;
based on detecting the ineffective stimulation, delivering a stimulation pulse via a new subset of electrodes from the multiple electrodes of the stimulation lead;
detecting effective stimulation of the phrenic nerve from the stimulation pulse via the new subset of electrodes; and
based on detecting effective stimulation of the phrenic nerve, delivering subsequent simulation pulses via the new subset of electrodes.

2. The phrenic nerve stimulation system of claim 1, wherein the stimulation source is a ventilator.

3. The phrenic nerve stimulation system of claim 1, wherein:
the stimulation pulse delivered via the initial subset of electrodes is delivered according to stimulus parameters; and
the stimulation pulse delivered via the new subset of electrodes is delivered according to the stimulus parameters.

4. The phrenic nerve stimulation system of claim 1, wherein detecting the ineffective stimulation includes comparing one or more respiratory metrics, for diaphragm movement caused by the stimulation pulse via an initial subset of electrodes, to one or more thresholds.

5. The phrenic nerve stimulation system of claim 1, wherein the initial subset of electrodes and new subset of electrodes are positioned on a deformable segment of the stimulation lead.

6. A computer-implemented method for selecting electrodes of a stimulation lead, the computer-implemented method comprising:
selecting a first pair of electrodes of the stimulation lead;
delivering a first stimulation pulse via the first pair of electrodes;
detecting ineffective stimulation of a phrenic nerve by the first stimulation pulse;
based on detecting ineffective stimulation the phrenic nerve by the first stimulation pulse, selecting a second pair of electrodes of the stimulation lead;
delivering a second stimulation pulse via the second pair of electrodes;
detecting ineffective stimulation of the phrenic nerve by the second stimulation pulse;
based on detecting ineffective stimulation the phrenic nerve by the second stimulation pulse, selecting a third pair of electrodes of the stimulation lead; and
delivering a third stimulation pulse via the third pair of electrodes.

7. The method of claim 6, wherein the second pair of electrodes are positioned on a first side of the first pair of electrodes, and the third pair of electrodes are positioned on an opposite side of the first pair of electrodes.

8. The method of claim 6, wherein the first stimulation pulse is delivered according to first stimulus parameters, and the second stimulation pulse is delivered according to second stimulus parameters, the second stimulus parameters having a greater intensity than the first stimulus parameters.

9. The method of claim 6, wherein the third stimulation pulse is delivered according to first stimulus parameters having a first intensity, and the method further comprises:
detecting ineffective stimulation of the phrenic nerve by the third stimulation pulse; and
based on detecting ineffective stimulation the phrenic nerve by the second stimulation pulse, delivering a fourth stimulation pulse via the third pair of electrodes, the fourth stimulation pulse being delivered according to second stimulus parameters having a greater intensity than the first stimulus parameters.

10. The method of claim 9, further comprising:
detecting ineffective stimulation of the phrenic nerve by the fourth stimulation pulse; and
based on detecting ineffective stimulation the phrenic nerve by the fourth stimulation pulse, selecting a fourth pair of electrodes.

11. The method of claim 9, further comprising:
detecting effective stimulation of the phrenic nerve by the fourth stimulation pulse; and
based on detecting effective stimulation the phrenic nerve by the fourth stimulation pulse, delivering subsequent stimulation pulses via the third pair of electrodes according to the second stimulus parameters.

12. A computer-implemented method for selecting electrodes of a stimulation lead, the computer-implemented method comprising:
delivering a stimulation pulse via an initial subset of electrodes of the stimulation lead;
detecting ineffective stimulation of a phrenic nerve from the delivered stimulation via the initial subset of electrodes;
based on detecting the ineffective stimulation, delivering a stimulation pulse via a new subset of electrodes of the stimulation lead;
detecting effective stimulation of the phrenic nerve from the stimulation pulse via the new subset of electrodes; and
based on detecting effective stimulation of the phrenic nerve, delivering subsequent simulation pulses via the new subset of electrodes.

13. The computer-implemented method of claim 12, wherein detecting the ineffective stimulation includes comparing one or more respiratory metrics, for diaphragm movement caused by the stimulation pulse via an initial subset of electrodes, to one or more thresholds.

14. The computer-implemented method of claim 12, wherein the initial subset of electrodes and new subset of electrodes are positioned on a deformable segment of the stimulation lead.

15. The computer-implemented method of claim 12, wherein the initial subset of electrodes are positioned on a first deformable segment of the stimulation lead and new subset of electrodes are positioned on a second deformable segment of the stimulation lead.
